(19) United States
(12) Patent Application Publication
(10) Pub. No.: US 2011/0060196 A1
(43) Pub. Date: Mar. 10, 2011

(54) Flexible Mounting Unit and Cover for a Medical Device

(75) Inventor: Gary Ashley Stafford, Hayward, CA (US)

(73) Assignee: Abbott Diabetes Care Inc., Alameda, CA (US)

(21) Appl. No.: 12/873,302

(22) Filed: Aug. 31, 2010

Related U.S. Application Data
(60) Provisional application No. 61/238,483, filed on Aug. 31, 2009.
(60) Provisional application No. 61/238,537, filed on Aug. 31, 2009.

(51) Int. Cl. A61B 5/00; H01Q 15/02

(52) U.S. Cl. 600/309; 343/909

(57) ABSTRACT

Generally, embodiments of the present disclosure are directed to an adhesive mounting unit that is formed from a nonwoven polymeric material that is breathable, flexible, waterproof and durable. Additionally, the adhesive mounting unit of the present disclosure includes a retention mechanism that is insert molded directly into or on the mounting unit such that the mounting unit and retention mechanism form a single unitary structure.

### RELATED APPLICATIONS

The present application claims the benefit of U.S. Provisional Patent Application No. 61/238,537, entitled “Cover for a Mounting Unit”, filed on Aug. 31, 2009, and U.S. Provisional Patent Application No. 61/238,483, entitled “Flexible Mounting Unit for a Medical Device”, filed on Aug. 31, 2009, the disclosures of each of which are herein incorporated by reference in their entirety for all purposes.

### BACKGROUND

Diabetes Mellitus is an incurable chronic disease in which the body does not produce or properly utilize insulin. Insulin is a hormone produced by the pancreas that regulates blood sugar (glucose). In particular, when blood sugar levels rise, e.g., after a meal, insulin lowers the blood sugar levels by facilitating blood glucose to move from the blood into the body cells. Thus, when the pancreas does not produce sufficient insulin (a condition known as Type I Diabetes) or does not properly utilize insulin (a condition known as Type II Diabetes), the blood glucose remains in the blood resulting in hyperglycemia or abnormally high blood sugar levels.

The vast and uncontrolled fluctuations in blood glucose levels in people suffering from diabetes cause long-term, serious complications. Some of these complications include blindness, kidney failure, and nerve damage. Additionally, it is known that diabetes is a factor in accelerating cardiovascular diseases such as atherosclerosis (hardening of the arteries), leading to stroke, coronary heart disease, and other diseases. Accordingly, one important and universal strategy in managing diabetes is to control blood glucose levels.

The first step in managing blood glucose levels is testing and monitoring blood glucose levels by using conventional techniques, such as drawing blood samples, applying the blood to a test strip, and determining the blood glucose level using colorimetric, electrochemical, or photometric test meters. Another more recent technique for monitoring blood glucose levels is by using a continuous or automatic glucose monitoring system, such as for example, the FreeStyle Navigator® Continuous Glucose Monitoring System, manufactured by Abbott Diabetes Care Inc. Unlike conventional blood glucose meters, continuous analyte monitoring systems employ an insertable or implantable sensor, which continuously detects and monitors blood glucose levels.

Generally, the implantable sensor is attached to the body of a user by a mounting structure. Conventionally, mounting structures are formed from rigid plastic material having adhesive to attach to a user's skin for a period of days. The long term wear of such mounting structures can not only irritate the skin but also can become uncomfortable, especially for active people. In this regard, there is a need for a mounting structure that is comfortable to wear and easy to adhere to the skin. As the insertion of the sensor typically requires piercing the skin, it would also be beneficial for such mounting unit to be adapted for administration of a medicament to treat any infection or other irritation associated with sensor implantation. Further, there is a need for a mount cover to enclose the sensor and/or an associated transmitter to protect them from becoming dislodged and/or contaminated from pollutants.

### INCORPORATED BY REFERENCE

The following patents, applications and/or publications are incorporated herein by reference for all purposes: U.S. Pat. Nos. 4,545,382; 4,711,245; 5,262,035; 5,262,305; 5,264,104; 5,320,715; 5,356,786; 5,509,410; 5,543,326; 5,593,852; 5,601,435; 5,628,890; 5,820,551; 5,822,715; 5,899,855; 5,918,603; 6,071,391; 6,103,033; 6,120,676; 6,121,009; 6,134,461; 6,143,164; 6,144,837; 6,161,095; 6,175,752; 6,270,455; 6,284,478; 6,299,757; 6,338,790; 6,377,894; 6,461,496; 6,503,381; 6,514,460; 6,514,718; 6,540,891; 6,560,471; 6,579,690; 6,591,125; 6,592,745; 6,600,997; 6,605,200; 6,605,201; 6,616,819; 6,618,934; 6,650,471; 6,654,625; 6,676,816; 6,730,200; 6,736,957; 6,746,582; . 6,749,740; 6,764,581; 6,773,671; 6,881,551; 6,893,545; 6,932,892; 6,932,894; 6,942,518; 7,041,468; 7,167,818; and 7,299,082; U.S. Published Application Nos. 2004/0186365; 2005/0182306; 2006/0025662; 2006/0091006; 2007/0056858; 2007/0068807; 2007/0078322; 2007/0095661; 2007/0108048; 2007/0199818; 2007/0227911; 2007/0233013; 2008/0066305; 2008/0081977; 2008/0102441; 2008/0148873; 2008/0161666; 2008/0267823; and 2009/0054748; U.S. patent application Ser. Nos. 11/461,725; 12/131,012; 12/393,921, 12/242,823; 12/363,712; 12/495,709; 12/698,124; 12/698,129; 12/714,439; 12/794,721; and 12/842,013, and U.S. Provisional Application Nos. 61/317,243, 61/345,562, and 61/361,374.

### SUMMARY

The present disclosure is directed to a mounting unit that is physically associated with a sensor. In certain embodiments, the mounting unit is a component of an analyte monitoring system. In another embodiment, the mounting unit is coupleable to an insertion device. Additionally, the mounting unit can be adapted to treat infection or irritation of a user's skin by administering a medicament once the mounting unit has been secured to the skin of the user. In another aspect, the mounting unit may be configured to contain insulin for transdermal delivery.

In certain embodiments, the mounting unit includes upper and lower opposing surfaces. The lower surface includes adhesive such that the mounting unit is attachable to the skin of the user. The upper surface includes a seat to receive a medical device, such as, for example, an integrated on-body sensor assembly or a coupled sensor and transmitter. In this manner, the mount can be configured to receive and enable coupling of both the sensor and the transmitter, or to receive the integrated sensor assembly and housing. In certain embodiments, one or more retainer elements are insert molded into or on the upper surface of the mounting unit. The retainer elements are configured to engage and secure the medical device in the seat of the mounting unit.

In one aspect, the mounting unit includes a seat or reception mechanism configured from a depression formed in the body of the mounting unit. In this manner, the medical device is received in the seat when coupled to the mounting unit.

It is contemplated that the mounting unit can be formed in a variety of shapes, thicknesses, and sizes. For example, the mounting unit body can be configured as a circular, semicircular, oval, rectangular, or any other polygonal shape. Generally, the mounting unit is configured to accommodate the shape of the medical device received and retained as an on-body unit. Thus, the mounting unit can be configured to have any desired shape.

It is contemplated that various types of retainer elements can be employed in embodiments of the present disclosure. For example, the retainer elements can include any structure configured to engage and seat a medical device. Such examples include a railing disposed on or otherwise formed in the body of the mounting unit, a protrusion, (e.g., button) extending upwardly from the upper surface of the mounting unit, or flanges that are configured to engage a body of a transmitter.

In certain embodiments, the lower surface of the mounting unit includes an adhesive to attach to the wearer of the mount. The adhesive, for example, can be a pressure-sensitive adhesive. In such instances, the mounting unit further includes a liner to cover the adhesive during periods of non-use. The adhesive can be a breathable adhesive and/or biocompatible adhesive.

In yet other embodiments, the lower surface of the mounting unit includes a medicament, for example, in the form of an ointment disposed on at least a portion of the lower surface such that the ointment comes into direct contact with the skin of the user. It is contemplated that the ointment may be a medicament to reduce the pain of insertion of the sensor to the body. Further, the ointment may be an antiseptic, an analgesic or an anti-inflammatory medicament. Moreover, the mounting unit can be adapted to contain insulin for delivery to the wearer.

In certain embodiments, the mounting unit can further comprise at least one opening formed through the upper and lower surfaces of the body. In such embodiments, the opening can be configured to permit a medical device, such as a transmitter to contact the skin of a wearer when the transmitter is coupled to the mounting unit. In this regard, the transmitter can include a temperature probe or module such that the transmitter is in contact with the skin of the wearer and enables detection and/or recordation of the skin temperature.

In certain embodiments, the mounting unit is a component of a continuous, semi-continuous, or discrete analyte monitoring system, such as a glucose monitoring system. In some embodiments, the mounting unit is a component of a sensor inserter kit. In this manner, the mounting unit is configured to form an interlocking engagement with an inserter kit (e.g., an inserter with pre-loaded sensor).

Further, the present disclosure provides a protective cover assembly for the transmitter when the transmitter is coupled to the mounting unit. The cover can include a first end, a second end and a plurality of sidewalls extending between the first and second ends to define an enclosed container.

In certain embodiments, the mounting unit includes a seal disposed on the upper surface, and the cover includes a fastener configured to engage the seal. In another embodiment, the mounting unit and the cover are configured to define an interlocking relationship by using at least one mechanical fastener. For example, the mechanical fastener can be one of an interference-fit, press-fit, or snap-fit closure. In yet another embodiment, the interlocking relationship is defined by hooks and loops, hooks, latches, pins, springs, or clips disposed on each of the mounting unit and the protective cover.

In accordance with yet another aspect of the present disclosure, the mounting unit or the protective cover can further include a barrier to prevent pollutants from entering the enclosed mounting unit. In some instances, the barrier is proximate the engagement of the mounting unit and cover. For example, the barrier can be rubber, thermoplastic elastomer, urethane, or silicone material, and extend around the enclosure defined by the engaged protective cover and the mounting unit. The barrier can further provide a waterproof or dustproof enclosure between the mounting unit and protective cover.

In certain embodiments, the cover and/or the mounting unit may be formed from a radio frequency conducive material. For example, the RF conductive material can be a thermoplastic or thermoset polymer. In another example, the cover and/or mounting unit can be formed from a low durometer polymer. In yet another example, the cover and/or mounting unit can be formed from a Bluetooth® plastic (e.g., thermoplastics, that improve the performance of electronic devices for use with Bluetooth® wireless communications protocol).

In another aspect of the present disclosure, the cover includes a radio frequency transmission booster. For example, the radio frequency transmission booster can include an antenna disposed within the material of the cover.

These and other objects, features and advantages of the present disclosure will become more fully apparent from the following detailed description of the embodiments, the appended claims and the accompanying drawings.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 illustrates a block diagram of a data monitoring and management system according to embodiments of the present disclosure;

FIG. 2 illustrates a mounting unit having a retention element molded into the body of the mounting unit according to embodiments of the present disclosure;

FIG. 3 illustrates a mounting unit having a retainer element molded into the body of the mounting unit according to another embodiment of the present disclosure;

FIG. 4 illustrates a mounting unit having mounting portion molded into the body of the mounting unit according to another embodiment of the present disclosure;

FIG. 5 illustrates a mounting unit having one or more opposing flanges molded into the body of the mounting unit according to another embodiment of the present disclosure;

FIG. 6 illustrates a mounting unit in accordance with another embodiment of the present disclosure; and

FIG. 7 illustrates a mount cover for use with a mounting unit according to embodiments of the present disclosure.

### DETAILED DESCRIPTION

Before the present disclosure is described in detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The figures shown herein are not necessarily drawn to scale, with some components and features being exaggerated for clarity.

Embodiments of the present disclosure are directed to an adhesive mounting unit that is formed from a nonwoven polymeric material that is breathable, flexible, waterproof and durable. As will be described in greater detail below, the adhesive mounting unit may be formed in various shapes and with varying thicknesses. Additionally, a device retention mechanism may be insert molded directly in or onto the adhesive mounting unit thus forming a single unitary structure.

FIG. 1 illustrates a data monitoring and management system such as, for example, analyte (e.g., glucose) monitoring system 100 in accordance with embodiments of the present disclosure. The analyte monitoring system 100 includes a sensor 101, a transmitter unit 102 coupleable to the sensor 101, and a primary receiver unit 104 which is configured to communicate with the transmitter unit 102 via a bi-directional communication link 103. The primary receiver unit 104 may be further configured to transmit data to a data processing terminal 105 for evaluating the data received by the primary receiver unit 104. Moreover, the data processing terminal 105 in one embodiment may be configured to receive data directly from the transmitter unit 102 via a communication link which may optionally be configured for bi-directional communication. Accordingly, transmitter unit 102 and/or receiver unit 104 may include a transceiver.

Also shown in FIG. 1 is an optional secondary receiver unit 106 which is operatively coupled to the communication link and configured to receive data transmitted from the transmitter unit 102. Moreover, as shown in the Figure, the secondary receiver unit 106 is configured to communicate with the primary receiver unit 104 as well as the data processing terminal 105. Indeed, the secondary receiver unit 106 may be configured for bidirectional wireless communication with each or one of the primary receiver unit 104 and the data processing terminal 105. In one embodiment of the present disclosure, the secondary receiver unit 106 may be configured to include a limited number of functions and features as compared with the primary receiver unit 104. As such, the secondary receiver unit 106 may be configured substantially in a smaller compact housing or embodied in a device such as a wrist watch, pager, mobile phone, Personal Digital Assistant (PDA), for example. Alternatively, the secondary receiver unit 106 may be configured with the same or substantially similar functionality as the primary receiver unit 104. The receiver unit may be configured to be used in conjunction with a docking cradle unit, for example for one or more of the following or other functions: placement by bedside, for re-charging, for data management, for night time monitoring, and/or bidirectional communication device.

In one aspect sensor 101 may include two or more sensors, each configured to communicate with transmitter unit 102. Furthermore, while only one, transmitter unit 102, communication link 103, and data processing terminal 105 are shown in the embodiment of the analyte monitoring system 100 illustrated in FIG. 1, in certain embodiments, the analyte monitoring system 100 may include one or more sensors, multiple transmitter units 102, communication links 103, and data processing terminals 105. Moreover, within the scope of the present disclosure, the analyte monitoring system 100 may be a continuous monitoring system, or semi-continuous, or a discrete monitoring system. In a multi-component environment, each device is configured to be uniquely identified by each of the other devices in the system so that communication conflict is readily resolved between the various components within the analyte monitoring system 100.

In certain embodiments, the sensor 101 is physically positioned in or on the body of a user whose analyte level is being monitored. The sensor 101 may be configured to continuously sample the analyte level of the user and convert the sampled analyte level into a corresponding data signal for transmission by the transmitter unit 102. In certain embodiments, the transmitter unit 102 may be physically coupled to the sensor 101 so that both devices are integrated in a single housing and positioned on the user's body. The transmitter unit 102 may perform data processing such as filtering and encoding on data signals and/or other functions, each of which corresponds to a sampled analyte level of the user, and in any event transmitter unit 102 transmits analyte information to the primary receiver unit 104 via the communication link 103. Additional detailed description of the continuous analyte monitoring system, its various components including the functional descriptions of the transmitter are provided in but not limited to U.S. Pat Nos. 6,134,461, 6,175,752, 6,121,611, 6,560,471, and 6,746,582, and U.S. Patent Publication No. 2008/0278332 and elsewhere, the disclosures of each of which are incorporated herein by reference for all purposes.

As described above, in certain embodiments, a transmitter unit 102 may be physically coupled to the sensor 101 so that both devices are integrated in a single housing and positioned and secured, via an adhesive, on the user's body. However, as the housing is secured directly to the user's skin, the user's movement may be restricted as certain movements may cause the adhesive to pull on the skin causing discomfort to the user. Additionally, the user's skin may become irritated or sensitive from being exposed to the adhesive for a prolonged period of time.

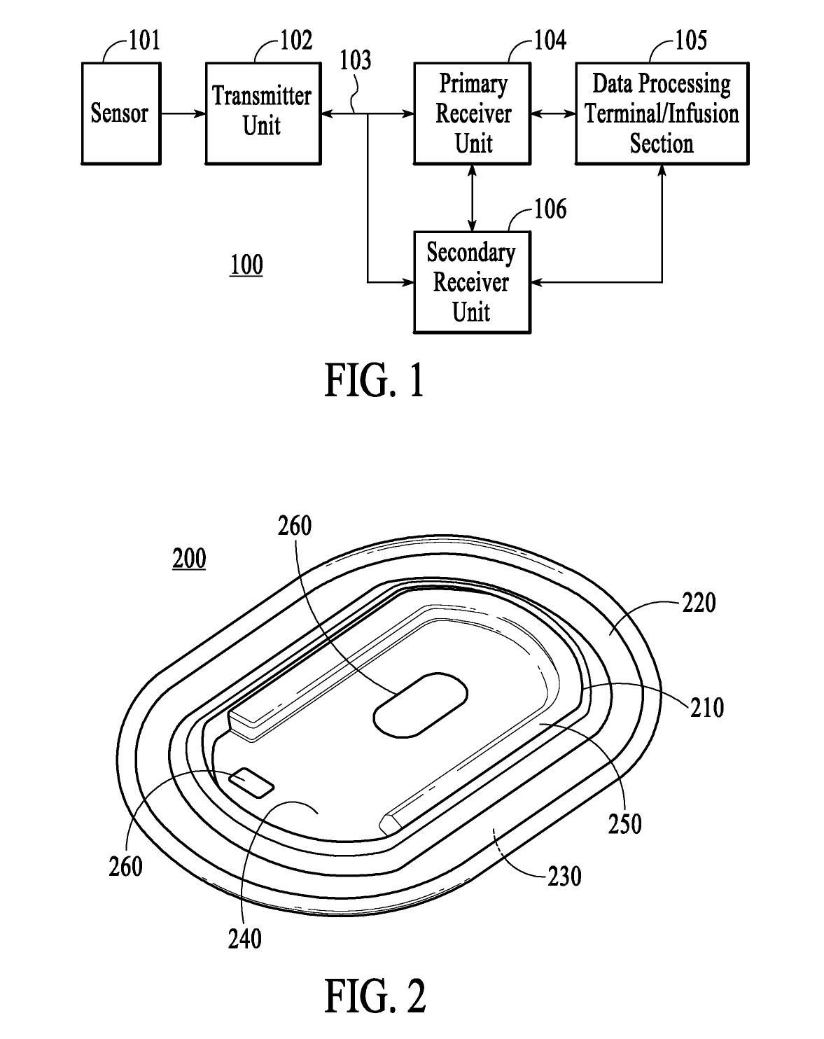

FIG. 2 illustrates a mounting unit 200 having mounting portion 210 molded into the body of the mounting unit 200 according to embodiments of the present disclosure. In certain embodiments, the mounting unit 200 is formed of a nonwoven polymeric material, such as, for example, styrenic block copolymers (SBCs) that are breathable, flexible, waterproof and durable. In certain embodiments, the mounting unit 200 may be bendable such that the body of the mounting unit 200 may be folded against itself. Other exemplary materials that may be used to form the mounting unit 200 include thermoplastic or thermoset polymers, including urethane and silicone.

As will be described in greater detail below, the mounting unit 200 of the present disclosure, in certain embodiments, comprises a patch that is molded into a desired form using the polymeric material described above. In this manner, the patch can be formed from various polymeric substrates by conventional injection molding techniques, insert molding techniques, in which the polymeric substrates are injected into a mold that contains a preplaced mechanism (e.g., mounting portion 210), or by using an overmold process in which rigid plastic or rubber is directly molded onto the patch to form the mounting portion 210. In certain embodiments, the patch may be molded into a plurality of shapes and thicknesses. For example, the patch may be molded in a contoured fashion such as to follow the contour of a body part (e.g., arm, abdomen, etc.) of a user onto which the mounting unit 200 will be placed. Thus, when the mounting unit 200 is placed on the body part of the user, the mounting unit 200 substantially conforms to the curvature of the user's body. The patch may be molded into a circle, square, rectangle, oval or any other polygonal shape.

As described above, in certain embodiments, during the molding process, the mounting portion 210 is insert molded directly into or onto the patch, thereby forming a single unitary structure. Further, because the patch may be molded into different shapes using the nonwoven polymeric material, the mounting unit 200 may be more flexible and provide additional comfort to the user.

Referring still to FIG. 2, in certain embodiments, the mounting unit 200 comprises a body having an upper surface 220 and a lower surface 230. In certain embodiments, upper surface 220 includes the mounting portion 210. In one aspect, the mounting portion 210 is comprised of a rigid member, such as a rigid polymer or rubber that is molded directly into or on the upper surface 220 of the mounting unit 200. The mounting portion 210 is configured to receive and retain a device, such as a transmitter (e.g., transmitter unit 102 (FIG. 1)) using the rigid members of the mounting portion 210. As such, in one aspect, the shape or configuration of the mounting unit 200 and/or the mounting portion 210 is shaped in such a way as to complement the device that is to be retained by the mounting unit 200.

In certain embodiments, the mounting unit 200 can also have fastening material disposed at least partially on the lower surface 230 of the mounting unit 200, such as, for example, an adhesive material. Thus, the mounting unit 200 can be attached to the user's skin with the adhesive. In certain embodiments, the mounting unit 200 may be configured as a transdermal patch configured to deliver a specific dose of medication through the skin and into the bloodstream of the user. In such embodiments, the lower surface 230 of the mounting unit 200 includes, among others, an antiseptic agent, an analgesic agent, an antibacterial agent, an anti-inflammatory agent, or insulin. Thus, the medicament disposed on the lower surface 230 may be controllably released into the bloodstream of the user when the mounting unit 200 is placed on the skin of the user.

In certain embodiments and as shown in FIG. 2, the mounting portion 210 of the mounting unit 200 includes a retainer element 250 configured as an upwardly extending railing which is generally disposed about a perimeter of a seat 240 disposed on the mounting unit 200. The retainer element 250 is configured to engage a device, such as transmitter unit 102 (FIG. 1), that has a complementary and corresponding engagement element such that the retainer element 250 engages and releaseably or fixedly retains the device.

In certain embodiments, the mounting unit 200 may also include one or more apertures 260 disposed through the upper surface 220 and the lower surface 230 of the mounting unit 200. In certain embodiments, the apertures 260 may be configured to permit portions of a device, such as transmitter unit 102 (FIG. 1), to couple to the mounting unit 200 to contact the skin of the user. For example, if the device is a transmitter having a temperature unit configured to monitor the temperature of the user's skin proximate to the sensor insertion site, the temperature unit may pass through one of the apertures 260 so as to directly contact the skin of the user. In another embodiment, the one or more apertures 260 may be configured to enable at least a portion of a transcutaneous sensor, such as, for example, sensor 101 (FIG. 1) to pass therethrough and pierce the skin of the user.

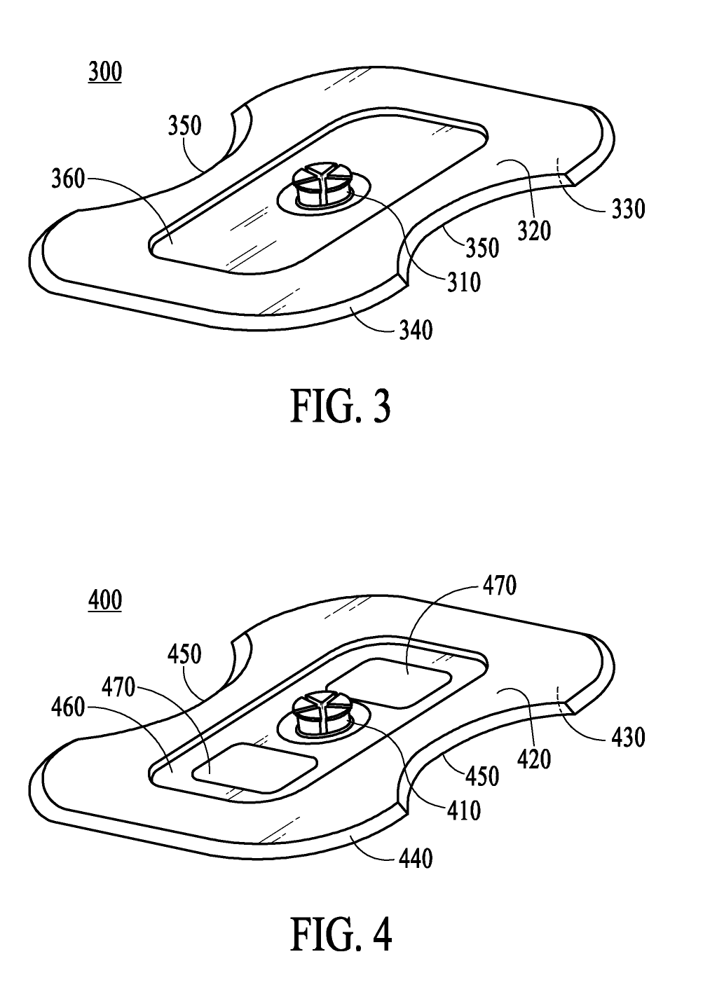

FIG. 3 illustrates a mounting unit 300 having a retainer element 310 molded into the body of the mounting unit 300 according to another embodiment of the present disclosure. In certain embodiments, the retainer element 310 is insert molded into or onto the mounting unit 300 such as was described above with respect to FIG. 2. As shown in FIG. 3, the mounting unit 300 includes an upper surface 320 and lower surface 330 having a tapered or beveled edge 340 between the surfaces. In certain embodiments, the tapered or beveled edge 340 is configured to provide a smooth transition between the skin of the user and the mounting unit 300. Such a transition may prevent clothing of a user that covers the mounting unit 300 from becoming snagged or otherwise caught on an edge of the mounting unit 300. As with the mounting unit 200 of FIG. 2, mounting unit 300, in certain embodiments, may be comprised of a nonwoven polymeric adhesive patch molded into a desired shape and thickness.

In one aspect, the mounting unit 300 further includes first and second opposing lateral sides 350, each of which have a curved configuration that define an overall generally sinusoidal configuration of the mounting unit 300. In certain embodiments, the sinusoidal configuration may enable the mounting unit 300 to be more flexible when attached to the skin of the user which may provide additional comfort to the user.

As shown in FIG. 3, in certain embodiments, the mounting unit 300 also includes a seat 360 configured to receive and retain a device, such as, for example, a transmitter (e.g., transmitter unit 102 (FIG. 1)), when the device is coupled to the mounting unit 300. In certain embodiments, the seat 360 has a depressed configuration such that seat 350 has a lower profile than the upper surface 320 of the mounting unit 300. As the seat 360 has a lower profile than the upper surface 320 of the mounting unit 300, when the device is coupled to the mounting unit 300, the overall profile of the device and mounting unit 300 may be reduced.

In certain embodiments, a retainer element 310 is disposed in the seat 360 of the mounting unit 300. In one aspect, the retainer element 310 has circular configuration with one or more flexible gaps defined throughout the retainer element 310. Accordingly, the device, such as transmitter unit 102 (FIG. 1), may be configured to releaseably engage the retainer element 310 of the mounting unit 300 via a female receptacle provided on the underside of the device. In one aspect, the retainer element 310 has an opening through which at least a portion of a sensor, such as, for example, sensor 101 (FIG. 1), may be placed so as to enable the portion of the sensor to be transcutaneously positioned. In one aspect, because the device is connected to only a portion of the mounting unit 300 via the retainer element 310, the mounting unit 300 may be more flexible as the flexibility of the mounting unit 300 is not restricted by having the entire device secured to the mounting unit 300. As the mounting unit 300 is more flexible with only a portion of the device attached, the user may be provided with a wider range of movement and/or additional comfort when moving the body part on which the mounting unit 300 is attached.

FIG. 4 illustrates a mounting unit 400 having mounting portion 410 molded into the body of the mounting unit 400 according to another embodiment of the present disclosure. In certain embodiments, the mounting portion 410 is insert molded into or onto the mounting unit 400 such as was described above. Additionally, the mounting unit 400 may be comprised of a nonwoven polymeric adhesive patch.

As shown in FIG. 4, mounting unit 400 has a similar configuration of mounting unit 300 (FIG. 3) including an upper surface 420, a lower surface 430, and a tapered or beveled edge 440 between the surfaces. Additionally, mounting unit 400 also includes first and second opposing lateral sides 450, each of which have a curved configuration that defines an overall generally sinusoidal configuration of the mounting unit 400. As with mounting unit 300, mounting unit 400 also includes a seat 460 disposed on the upper surface 420 of the mounting unit 400 which is configured to help reduce the overall profile of the mounting unit 400 when a device, such as, for example a transmitter, is coupled to the mounting unit 400 via the mounting portion 410.

In one aspect, mounting unit 400 includes a plurality of apertures 470 that are disposed axially and laterally from the mounting portion 410. As discussed above with respect to FIG. 2, the apertures 470 may be configured to allow a sensor, or temperature unit of a transmitter, such as transmitter unit 102 (FIG. 1), to pass through the mounting unit 400 and contact the skin of a user. As was described above, the temperature unit may be configured to measure the temperature of the user's skin around the sensor insertion site.

As with the configuration of mounting unit 300 (FIG. 3) described above, mounting unit 400 enables a user to connect only a portion of the transmitter unit 102 to the mounting unit 400. As such, the mounting unit 400 may be more flexible as the flexibility of the mounting unit 400 is not restricted by having all or substantially all of the transmitter unit 102 secured to the mounting unit 400.

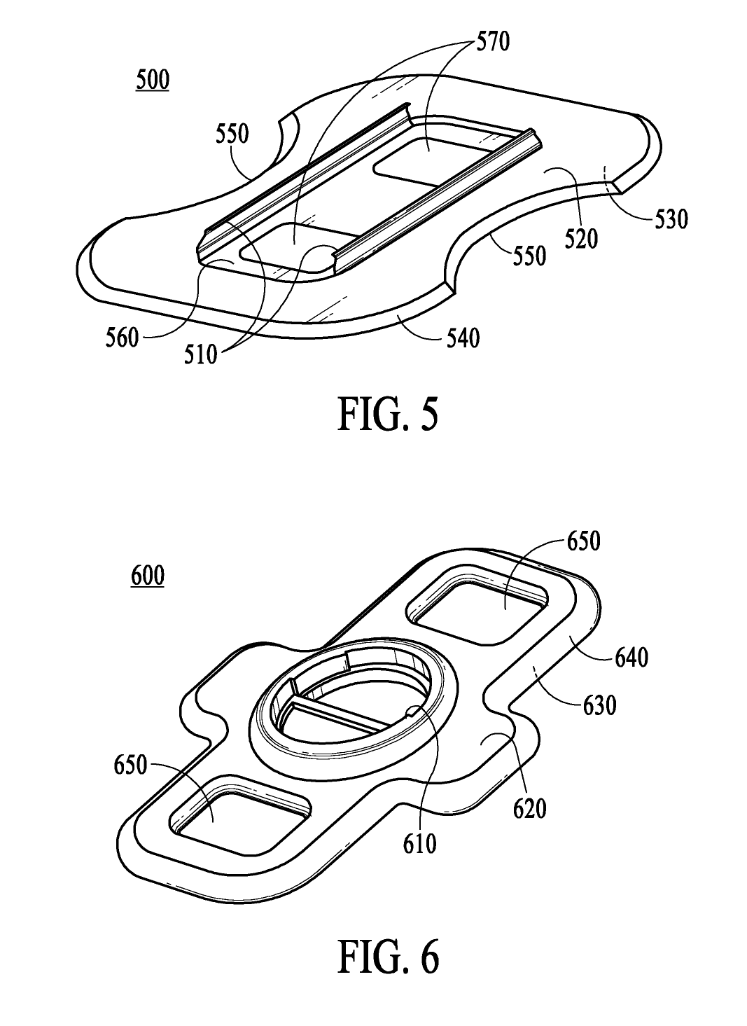

FIG. 5 illustrates a mounting unit 500 having one or more opposing flanges 510 molded into the body of the mounting unit 500 according to another embodiment of the present disclosure. In certain embodiments, the mounting unit 500 may be comprised of a nonwoven polymeric adhesive patch having the opposing flanges 510 being insert molded into or onto the mounting unit 500 such as was described above. As shown in FIG. 5, the opposing flanges 510 have an upwardly extending length with a generally curved configuration so as to retain a device, such as, for example a transmitter unit 102, a sensor 101, or other such device, in the seat 560 of the mounting unit 500.

In certain embodiments, the mounting unit 500 includes an upper surface 520, a lower surface 530, and a tapered or beveled edge 540 between the surfaces. Additionally, mounting unit 500 also includes first and second opposing lateral sides 550, each of which have a curved configuration that defines an overall generally sinusoidal configuration of the mounting unit 500. In one aspect, mounting unit 500 also includes a plurality of apertures 570 configured to allow a sensor or a temperatures module of a transmitter to pass through the mounting unit 500 and contact the skin of a user.

In certain embodiments, the opposing flanges 510 of the mounting unit 500 may be configured to engage a housing of a transmitter unit 102 in a snap-fit engagement. Thus, because the mounting unit 500 is flexible, a manual force may be applied to at least one of the opposing flanges 510 to cause the flange to temporarily move away from the other flange. Once the opposing flanges 510 have temporarily moved in an outward direction, the device may be placed in between the opposing flanges 510. When the manual force is removed, the opposing flanges 510 return to their original configuration thus securing the device. In another aspect, the opposing flanges 510 may act as a track onto which a housing of the transmitter unit 102 may slideably engage. Such a configuration as described above may be beneficial to user that is participating in various activities (e.g., athletic activities) as the device may be more securely retained and is less likely to become dislodged or disconnected from the user during the particular activity.

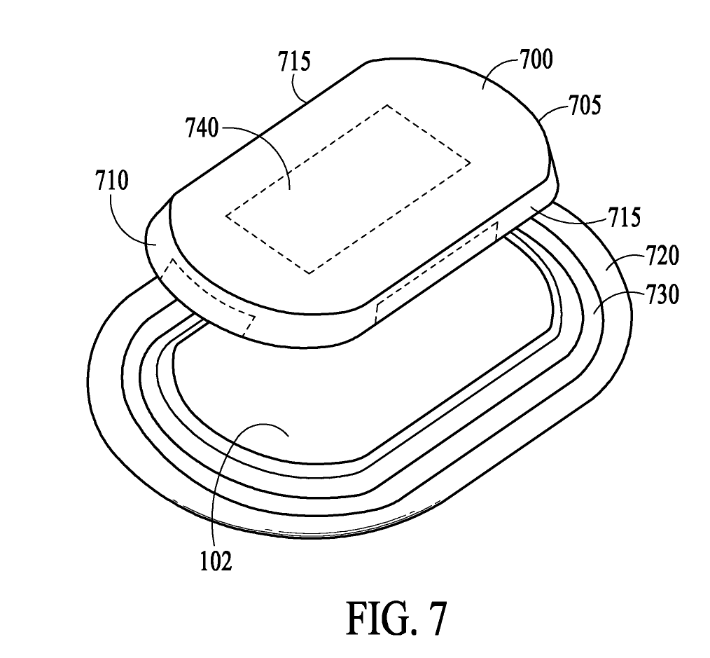

FIG. 6 illustrates a mounting unit 600 in accordance with another embodiment of the present disclosure. As shown in FIG. 6, the mounting unit 600 is molded into a cross-type configuration having first and second arms arranged perpendicular manner. The mounting unit 600 also includes an upper surface 620, a lower surface 630, and a tapered or beveled edge 640 disposed between the surfaces. In one aspect, mounting unit 600 also includes a plurality of apertures 650 configured to allow a sensor or a temperatures module of a transmitter to pass through the mounting unit 600 and contact the skin of a user. Additionally, the mounting unit 600 may be comprised of a nonwoven polymeric adhesive patch such as was described above.

In certain embodiments, the mounting unit 600 includes a circular ratchet mechanism 610 configured to releaseably retain a device, such as, for example, a sensor 101 or transmitter unit 102 (FIG. 1). In certain embodiments, the ratchet mechanism 610 may be configured to releaseably couple an integrated sensor assembly having electronics for communication with a receiver, such as, for example, primary receiver unit 104 (FIG. 1). For example, the integrated sensor assembly may have a rounded configuration and the ratchet mechanism 610 may be configured to receive at least a portion of the integrated sensor assembly. Once the integrated sensor assembly has been inserted into the ratchet mechanism 610, the integrated sensor assembly may be rotated a predetermined number of degrees such that the ratchet mechanism 610 is secured to the mounting unit 600. Once the integrated sensor assembly has been secured to the mounting unit 600, the sensor of the integrated assembly may be inserted into the skin of the user. Additional detailed description of an integrated sensor assembly is provided in U.S. Patent Publication No. 2010/0198034, the disclosure of which is incorporated by reference for all purposes.

In certain embodiments, one or more of the mounting units described above may be configured to receive an insertion device configured to transcutaneously position a sensor into the skin of a user. In such embodiments, after deployment of the sensor into the user's body, a medical device, such as a sensor assembly, can be engaged to the mounting unit. In another aspect, the mounting unit can be a component of the insertion device. In this manner, the mounting unit is configured to receive a sensor assembly and is also configured to engage the insertion device.

In another aspect, the mounting unit is used with an analyte monitoring system, such as, for example analyte monitoring system 100 (FIG. 1). In this manner, the mounting unit retains sensor 101 and transmitter unit 102, such that after implantation of sensor 101 into a user's body, the sensor 101 is coupled to transmitter unit 102 and the transmitter unit 102 is coupled to the mounting unit. In still yet another embodiment, the mounting unit can be coupled to an integrated sensor assembly such that the housing of the sensor assembly is coupled to the mounting unit.

Once the mounting unit has been placed on the user's body and a device, such as, for example, transmitter unit 102, has been secured to the mounting unit, in certain embodiments there is risk that the transmitter unit 102 may become dislodged and/or damaged based on various activities of the user. Furthermore, the device may become dirty or contaminated with pollutants that may affect the overall functionality of the device. Accordingly, FIG. 7 illustrates a mount cover 700 for a mounting unit according to embodiments of the present disclosure. In certain embodiments, the mount cover 700 is provided for a mounting unit 720. In certain embodiments, mounting unit 720 may be similar to one or more of the mounting units described above with respect to FIGS. 2-6.

In certain embodiments, the mount cover 700 is configured to provide protection to a device, such as, for example transmitter unit 102 (FIG. 1) that is coupled to the mounting unit 720. In this regard, the mount cover 700 can protect the transmitter unit 102 from moisture, impact, dust and other potential hazards which may cause the transmitter unit 102 to become disconnected from sensor 101 and/or cause an interruption or disconnection from a signal of a receiver (e.g., primary receiver unit 104 (FIG. 1)).

In certain embodiments, the mount cover 700 includes a first end 705, a second end 710 and a plurality of sidewalls 715 extending between the first 705 and second 710 ends that define an enclosed container. The mount cover 700 is configured to sealingly engage and enclose a transmitter unit 102, or other device, disposed on the mounting unit 720. In certain embodiments, various engagement means can be employed to seal the mount cover 700 to the mounting unit 720. For example, the mount cover 700 and the mounting unit 720 can be configured to define a snap-on, interference fit, or press-fit closure. In another embodiment, the mount cover 700 and the adhesive mounting unit 720 include mechanical fasteners, such as hooks, latches, pins, springs, clips, and the like. In still yet other embodiments, chemical fasteners can be used such as resealable and/or reusable adhesives. Thus, the mount cover 700 can be releaseably attached to the mounting unit 720 and be reused.

In certain embodiments, a sealing barrier member 730 may be disposed on the mount cover 700 and/or the upper surface of the mounting unit 720. In one aspect, the sealing barrier member 730 is configured to overlay or otherwise cover the closure between the mounting unit 720 and the mount cover 700 to further protect transmitter unit 102 from environmental pollutants, such as moisture and dust, and the like. Additionally, the cover mount can facilitate maintenance of suitable operating temperatures of the transmitter unit 102 when used in extreme environments.

In certain embodiments, the mount cover 700 includes a radio frequency (RF) transmission booster 740, such as, for example, an antenna or amplifier, disposed within the mount cover 700 to increase the signal range of the transmitter unit 102. For example, the transmission booster 740 can be insert molded directly into or onto the mount cover 700 such as was described above. In another embodiment, the transmission booster 740 , or can be applied by an adhesive (e.g., pressure sensitive adhesive) to the mount cover 700.

In certain embodiments, the mount cover 700 can be formed from injection molded or thermoformed flexible polymers and/or malleable metals that do not interfere with radio frequency transmissions. Such materials may include elastomers, including thermoplastic and thermoset polymers. Such materials can include modified electrical and dielectrical properties to enhance data or signal transmission and reception. For example, polycarbonates, polyphenylene sulfide (PPS), polycarbonate/ABS blends, ABS, and thermoplastic elastomer (TPE) compounds can be used.

In the manner described above, a mounting unit is provided that is breathable, flexible, waterproof and durable so as to provide additional comfort to a user when the mounting unit is secured to the user's skin. Additionally, the mounting unit may be molded into a number of different sizes and shapes that may better fit each user's unique body shape and size. For example, the mounting unit may be provided in a first size, shape and thickness for a child user and be provided in a second size, shape and thickness for an adult user. Additionally, the mounting unit may be configured to secure any number of devices having various shapes and sizes.

In a further embodiment of the present disclosure, a device cover is provided to protect the device from pollutants and/or impacts that may affect the overall functionality of the device. In certain embodiments, the device cover is configured to be secured to any one of the mounting units discussed above with respect to FIGS. 2-6.

Certain embodiments of the present disclosure include a mounting unit for mounting a transmitter unit 102 and coupled sensor 101 on the skin of a patient. The mounting unit may be shaped and constructed of materials to facilitate flexibility and accordingly increase comfort to the user. Additional embodiments include a mount cover to protect or further adhere the transmitter unit 102 and the mounting unit.

In certain aspects of the present disclosure, a mounting unit may include a contoured patch formed of a nonwoven polymeric material having a flexible body with an upper surface and a lower surface, wherein the upper surface has a seat in a depressed configuration with respect to the upper surface, and a retention mechanism disposed in the seat, wherein the retention mechanism is coupled to the body such that the retention mechanism and the flexible body form a single unitary structure, and wherein the retention mechanism is configured to releaseably engage a medical device.

In certain embodiments, the medical device may be a transmitter.

In certain embodiments, the medical device may be a sensor.

In certain embodiments, the medical device may be an integrated sensor assembly.

In certain embodiments, the retention mechanism may be insert molded into the seat.

In certain embodiments, the patch may be a transdermal patch.

Certain embodiments may include an adhesive disposed at least partially on the lower surface of the mounting unit.

Certain embodiments may include a medicament disposed at least partially on the lower surface of the mounting unit.

In certain aspects of the present disclosure, a mounting unit may include a contoured patch having a flexible body with an upper surface and a lower surface, wherein the upper surface has a seat in a depressed configuration with respect to the upper surface and a retention mechanism disposed in the seat such that the retention mechanism and the flexible body form a single unitary structure, wherein the retention mechanism is configured to releaseably retain a medical device, and further wherein the upper surface includes a retention seal disposed on the upper surface, and a cover configured substantially enclose the medical device and engage the retention seal disposed on the upper surface of the mounting unit.

In certain embodiments, the cover may include a fastener configured to engage the seal.

In certain embodiments, the cover may be formed from a radio frequency conductive material.

In certain embodiments, the cover may include a radio frequency transmission booster.

In certain embodiments, the radio frequency transmission booster may include an antenna disposed within the material of the cover.

In certain embodiments, the radio frequency transmission booster is insert molded into the cover.

In certain embodiments, the medical device may be an integrated sensor assembly.

In certain embodiments, the medical device may be a non-integrated sensor assembly.

In certain embodiments, the medical device may be a transmitter.

In certain embodiments, the body may be comprised of a nonwoven polymeric material.

In certain embodiments, the nonwoven polymeric material may be a styrenic block copolymer.

In certain embodiments, the retention mechanism may include at least one protrusion extending upwardly from the upper surface of the body.

Various other modifications and alterations in the structure and method of operation of this disclosure will be apparent to those skilled in the art without departing from the scope and spirit of the embodiments of the present disclosure. Although the present disclosure has been described in connection with particular embodiments, it should be understood that the present disclosure as claimed should not be unduly limited to such particular embodiments. It is intended that the following claims define the scope of the present disclosure and that structures and methods within the scope of these claims and their equivalents be covered thereby.

What is claimed is:

1. A mounting unit, comprising:
a contoured patch formed of a nonwoven polymeric material having a flexible body with an upper surface and a lower surface, wherein the upper surface has a seat in a depressed configuration with respect to the upper surface; and
a retention mechanism disposed in the seat, wherein the retention mechanism is coupled to the body such that the retention mechanism and the flexible body form a single unitary structure, and wherein the retention mechanism is configured to releaseably engage a medical device.

2. The mounting unit of claim 1, wherein the medical device is a transmitter.

3. The mounting unit of claim 1, wherein the medical device is a sensor.

4. The mounting unit of claim 1, wherein the medical device is an integrated sensor assembly.

5. The mounting unit of claim 1, wherein the retention mechanism is insert molded into the seat.

6. The mounting unit of claim 1, wherein the patch is a transdermal patch.

7. The mounting unit of claim 1, further comprising an adhesive disposed at least partially on the lower surface of the mounting unit.

8. The mounting unit of claim 1, further comprising a medicament disposed at least partially on the lower surface of the mounting unit.

9. A mounting unit, comprising:
a contoured patch having a flexible body with an upper surface and a lower surface, wherein the upper surface has a seat in a depressed configuration with respect to the upper surface and a retention mechanism disposed in the seat such that the retention mechanism and the flexible body form a single unitary structure, wherein the retention mechanism is configured to releaseably retain a medical device, and further wherein the upper surface includes a retention seal disposed on the upper surface; and
a cover configured substantially enclose the medical device and engage the retention seal disposed on the upper surface of the mounting unit.

10. The mounting unit of claim 9, wherein the cover includes a fastener configured to engage the seal.

11. The mounting unit of claim 9, wherein the cover is formed from a radio frequency conductive material.

12. The mounting unit of claim 9, wherein the cover includes a radio frequency transmission booster.

13. The mounting unit of claim 12, wherein the radio frequency transmission booster includes an antenna disposed within the material of the cover.

14. The mounting unit of claim 12, wherein the radio frequency transmission booster is insert molded into the cover.

15. The mounting unit of claim 9, wherein the medical device is an integrated sensor assembly.

16. The mounting unit of claim 9, wherein the medical device is a non-integrated sensor assembly.

17. The mounting unit of claim 9, wherein the medical device is a transmitter.

18. The mounting unit of claim 9, wherein the body is comprised of a nonwoven polymeric material.

19. The mounting unit of claim 18, wherein the nonwoven polymeric material is a styrenic block copolymer.

20. The mounting unit of claim 9, wherein the retention mechanism includes at least one protrusion extending upwardly from the upper surface of the body.
